(19) United States
(12) Patent Application Publication
(10) Pub. No.: US 2025/0002985 A1
(43) Pub. Date: Jan. 2, 2025

(54) USING APERTURES TO CAPTURE POLYNUCLEOTIDES ON PARTICLES

(71) Applicant: Illumina, Inc., San Diego, CA (US)

(72) Inventors: Gianluca Artioli, Cambridge (GB); Xavier von Hatten, Cambridge (GB); Alexandre Richez, Cambridge (GB); Wayne George, Haverhill (GB); Andrew Brown, Cambridge (GB); Istvan Kocsis, Cambridge (GB); Adam White, San Diego, CA (US)

(73) Assignee: Illumina, Inc., San Diego, CA (US)

(21) Appl. No.: 18/756,653

(22) Filed: Jun. 27, 2024

Related U.S. Application Data
(60) Provisional application No. 63/511,338, filed on Jun. 30, 2023.

(51) Int. Cl. C12Q 1/6844; C12Q 1/6806; C12Q 1/6834

(52) U.S. Cl. CPC C12Q 1/6844; C12Q 1/6806; C12Q 1/6834

(57) ABSTRACT

In some examples, a method of capturing a polynucleotide on a particle that includes a capture primer includes transporting the particle to a first aperture between a first fluidic compartment and a second fluidic compartment. The particle may be located in the first fluidic compartment, and the polynucleotide may be at least partially located in the second fluidic compartment. The method may include transporting the polynucleotide from the second fluidic compartment to the first fluidic compartment through the first aperture. The method may include hybridizing the polynucleotide to the capture primer.

### CROSS-REFERENCE TO RELATED APPLICATIONS

This application claims the benefit of U.S. Provisional Patent Application No. 63/511,338, filed on Jun. 30, 2023 and entitled “Using Apertures to Capture Polynucleotides on Particles,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2441-US.xml”, was created on Jun. 17, 2024 and is 8 KB in size.

### FIELD

This application generally relates to capturing polynucleotides.

### BACKGROUND

Cluster amplification is an approach to amplifying polynucleotides, for example for use in genetic sequencing. Target polynucleotides are captured by primers (e.g., P5 and P7 primers) coupled to a substrate surface in a flowcell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to using apertures to capture polynucleotides on particles. Devices for performing such capture also are disclosed.

Some examples herein provide a method of capturing a polynucleotide on a particle including a capture primer. The method may include transporting the particle to a first aperture between a first fluidic compartment and a second fluidic compartment. The particle is located in the first fluidic compartment, and the polynucleotide is at least partially located in the second fluidic compartment. The method may include transporting the polynucleotide from the second fluidic compartment to the first fluidic compartment through the first aperture. The method may include hybridizing the polynucleotide to the capture primer.

In some examples, the method further includes, after hybridizing the polynucleotide to the capture primer, transporting the particle away from the first aperture. In some examples, transporting the particle to the first aperture and away from the aperture includes flowing a fluid, in which the particle is suspended, through the first fluidic compartment and past the aperture.

In some examples, the method further includes synchronizing transport of the particle to the first aperture with transport of the polynucleotide through the first aperture. In some examples, the synchronizing includes: detecting transport of the polynucleotide through the first aperture; and controlling transport of the particle to the first aperture based on the detected transport of the polynucleotide through the first aperture. In some examples, the synchronizing includes: transporting the particle through a second aperture and into the first fluidic compartment; detecting transport of the particle through the second aperture; and controlling transport of the polynucleotide through the first aperture based on the detected transport of the particle through the second aperture. In some examples, the synchronizing includes: electronically controlling transport of the particle through a second aperture and into the first fluidic compartment; and electronically controlling transport of the polynucleotide through the first aperture and into the first fluidic compartment. In some examples, the second aperture includes a nanopore. In some examples, the second aperture has a diameter of about 50 nm to about 1000 nm.

In some examples, the particle has a diameter of about 50 nm to about 1000 nm.

In some examples, the first aperture includes a nanopore.

In some examples, the first aperture has a diameter of about 2 nm to about 20 nm.

In some examples, transporting the polynucleotide through the first aperture includes flowing a fluid, in which the polynucleotide is suspended, through the aperture and into the first fluidic compartment. In some examples, a plurality of polynucleotides are suspended in the fluid at a concentration of about 1 nM to about 100 nM.

In some examples, the polynucleotide is transported through the first aperture before hybridizing the polynucleotide to the capture primer. In some examples, before being transported through the first aperture, the polynucleotide is located entirely in the second fluidic compartment.

In some examples, the polynucleotide is transported through the first aperture in response to hybridizing the polynucleotide to the capture primer. In some examples, prior to hybridizing the polynucleotide to the capture primer, a first portion of the polynucleotide is located in the second fluidic compartment and a second portion of the polynucleotide is located in the first fluidic compartment. In some examples, responsive to hybridizing the polynucleotide to the capture primer, the first portion of the polynucleotide is transported from the second fluidic compartment into the first fluidic compartment through the first aperture. In some examples, the first portion of the polynucleotide retains the first portion of the polynucleotide in the second fluidic compartment. In some examples, the first portion of the polynucleotide includes a DNA loop. In some examples, when the polynucleotide is hybridized to the capture primer, force from the particle dissociates the DNA loop.

In some examples, the first portion of the polynucleotide is coupled to a structure that is at least partially located within the second fluidic compartment and retains the first portion of the polynucleotide in the second fluidic compartment. In some examples, the structure includes a DNA loop, a DNA hairpin, a cruciform folded double strand, or a dendrimer. In some examples, when the polynucleotide is hybridized to the capture primer, force from the particle dissociates the first portion of the polynucleotide from the structure, and the structure remains within the second fluidic compartment.

In some examples, the polynucleotide is transported through the first aperture after hybridizing the polynucleotide to the capture primer. In some examples, the capture primer extends through the aperture and hybridizes to the polynucleotide in the second fluidic compartment. In some examples, when the polynucleotide is hybridized to the capture primer, force from the particle pulls the polynucleotide through the first aperture and into the first fluidic compartment.

In some examples, the polynucleotide includes an adapter that is complementary to the capture primer.

In some examples, the capture primer includes an amplification primer.

In some examples, the particle includes a plurality of capture primers.

In some examples, the polynucleotide is single-stranded. In some examples, the polynucleotide is double-stranded.

Some examples herein provide another method of capturing a polynucleotide on a particle including a capture primer. The method may include transporting the particle to an aperture between a first fluidic compartment and a second fluidic compartment. The particle is located in the first fluidic compartment and the polynucleotide is coupled to a structure located in the second compartment. The method may include hybridizing the polynucleotide to the capture primer. The method may include dissociating the polynucleotide from the structure using a force applied by the particle.

Some examples herein provide another method of capturing a polynucleotide on a particle including a capture primer. The method may include transporting the particle to an aperture between a first fluidic compartment and a second fluidic compartment. The particle is located in the first fluidic compartment and the polynucleotide is located in the second compartment. The method may include extending the capture primer through the aperture. The method may include hybridizing the polynucleotide to the capture primer. The method may include generating an amplicon of the polynucleotide using the capture primer. The method may include transporting the amplicon from the second fluidic compartment to the first fluidic compartment through the aperture.

Some examples herein provide a method of generating a clonal cluster of a polynucleotide on a particle. The method may include capturing the polynucleotide on the particle using any of the foregoing methods. The method may include using a plurality of amplification primers on the particle to amplify the polynucleotide.

Some examples herein provide a device for capturing a polynucleotide on a particle including a capture primer. The device may include a first fluidic compartment; a second fluidic compartment; a first aperture defined through a wall between the first fluidic compartment and the second fluidic compartment; and a controller. The controller may be to transport the particle within the first fluidic compartment to the first aperture; and transport the polynucleotide from the second fluidic compartment to the first fluidic compartment through the first aperture so as to hybridize the polynucleotide to the capture primer.

In some examples, the controller further is to transport the particle away from the first aperture after the polynucleotide is hybridized to the capture primer. In some examples, the controller is to transport the particle to the first aperture and away from the aperture by flowing a fluid, in which the particle is suspended, through the first fluidic compartment and past the aperture.

In some examples, the controller further is to synchronize transport of the particle to the first aperture with transport of the polynucleotide through the first aperture. In some examples, the controller is to synchronize transport using operations including: detecting transport of the polynucleotide through the first aperture; and controlling transport of the particle to the first aperture based on the detected transport of the polynucleotide through the first aperture. In some examples, the controller is to synchronize transport using operations including: transporting the particle through a second aperture and into the first fluidic compartment; detecting transport of the particle through the second aperture; and controlling transport of the polynucleotide through the first aperture based on the detected transport of the particle through the second aperture. In some examples, the controller is to synchronize transport using operations including: electronically controlling transport of the particle through a second aperture and into the first fluidic compartment; and electronically controlling transport of the polynucleotide through the first aperture and into the first fluidic compartment.

In some examples, the second aperture includes a nanopore. In some examples, the second aperture has a diameter of about 50 nm to about 1000 nm.

In some examples, the particle has a diameter of about 50 nm to about 1000 nm.

In some examples, the first aperture includes a nanopore.

In some examples, the first aperture has a diameter of about 20 nm to about 200 nm.

In some examples, the controller is to transport the polynucleotide through the first aperture by flowing a fluid, in which the polynucleotide is suspended, through the aperture and into the first fluidic compartment. In some examples, a plurality of polynucleotides are suspended in the fluid at a concentration of about 1 nM to about 100 nM.

In some examples, the controller is to transport the polynucleotide through the first aperture before the polynucleotide is hybridized to the capture primer. In some examples, before being transported through the first aperture, the polynucleotide is located entirely in the second fluidic compartment.

In some examples, the polynucleotide is transported through the first aperture in response to hybridizing the polynucleotide to the capture primer. In some examples, prior to the polynucleotide being hybridized to the capture primer, a first portion of the polynucleotide is located in the second fluidic compartment and a second portion of the polynucleotide is located in the first fluidic compartment. In some examples, responsive to the polynucleotide hybridizing to the capture primer, the first portion of the polynucleotide is transported from the second fluidic compartment into the first fluidic compartment through the first aperture. In some examples, the first portion of the polynucleotide retains the first portion of the polynucleotide in the second fluidic compartment.

In some examples, the first portion of the polynucleotide includes a DNA loop. In some examples, when the polynucleotide is hybridized to the capture primer, force from the particle dissociates the DNA loop.

In some examples, the first portion of the polynucleotide is coupled to a structure that is at least partially located within the second fluidic compartment and retains the first portion of the polynucleotide in the second fluidic compartment. In some examples, the structure includes a DNA loop, a DNA hairpin, a cruciform folded double strand, or a dendrimer. In some examples, when the polynucleotide is hybridized to the capture primer, force from the particle dissociates the first portion of the polynucleotide from the structure, and the structure remains within the second fluidic compartment.

In some examples, the controller is to transport the polynucleotide after the polynucleotide is hybridized to the capture primer. In some examples, the capture primer extends through the aperture and hybridizes to the polynucleotide in the second fluidic compartment. In some examples, when the polynucleotide is hybridized to the capture primer, force from the particle pulls the polynucleotide through the first aperture and into the first fluidic compartment.

In some examples, the polynucleotide includes an adapter that is complementary to the capture primer.

In some examples, the capture primer includes an amplification primer.

In some examples, the particle includes a plurality of capture primers.

In some examples, the polynucleotide is single-stranded. In some examples, the polynucleotide is double-stranded.

Some examples herein provide another device for capturing a polynucleotide on a particle including a capture primer. The device may include a first fluidic compartment; a second fluidic compartment; a first aperture defined through a wall between the first fluidic compartment and the second fluidic compartment; and a controller. The controller may be to transport the particle within the first fluidic compartment to the first aperture so that the polynucleotide hybridizes with a capture primer coupled to a structure located in the second fluidic compartment, and so that the polynucleotide dissociates from the structure using a force applied by the particle.

Some examples herein provide another device for capturing a polynucleotide on a particle including a capture primer. The device may include a first fluidic compartment; a second fluidic compartment; a first aperture defined through a wall between the first fluidic compartment and the second fluidic compartment; and a controller. The controller may be to transport the particle within the first fluidic compartment to the first aperture so that the capture primer extends through the aperture to hybridize to the polynucleotide in the second fluidic compartment such that an amplicon of the polynucleotide is generated in the second compartment and the amplicon is transported from the second fluidic compartment to the first fluidic compartment through the first aperture.

Some examples herein provide a method of generating a clonal cluster of a polynucleotide on a particle. The method may include capturing the polynucleotide on the particle using any of the above devices; and using a plurality of amplification primers on the particle to amplify the polynucleotide.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1H schematically illustrate an example device and operations for using an aperture to capture a polynucleotide on a particle, and optionally also amplifying the captured polynucleotide using the particle.

FIG. 2 schematically illustrates an example device and operations for implementing operations such as described with reference to FIGS. 1A-1H.

FIG. 3 schematically illustrates another example device and operations for implementing operations such as described with reference to FIGS. 1A-1H.

FIG. 4 schematically illustrates another example device and operations for implementing operations such as described with reference to FIGS. 1A-1H.

FIG. 5 schematically illustrates another example device and operations for implementing operations such as described with reference to FIGS. 1A-1H.

FIG. 6 schematically illustrates another example device and operations for implementing operations such as described with reference to FIGS. 1A-1H.

FIG. 7 schematically illustrates another example device and operation for implementing operations such as described with reference to FIGS. 1A-1H.

FIG. 8 schematically illustrates another example device and operations for implementing operations such as described with reference to FIGS. 1A-1H.

FIG. 9 schematically illustrates another example device and operations for implementing operations such as described with reference to FIGS. 1A-1H.

### DETAILED DESCRIPTION

Examples provided herein are related to using apertures to capture polynucleotides on particles. Devices for performing such capture also are disclosed.

As provided herein, apertures may be used to control the manner in which particles are used to capture corresponding polynucleotides. More specifically, the apertures may be used to control the respective motion of the particles and/or the polynucleotides relative to one another such that particles respectively capture a single polynucleotide. The particles then may be used to amplify the respective captured single polynucleotide, so as to generate a substantially monoclonal cluster of amplicons using each such particle. The present methods and devices may be used to capture polynucleotides using any suitable type of particle, offering significant flexibility. Furthermore, because the present apertures may help to generate a relatively large yield of substantially monoclonal clusters, it is expected that the amount of waste in sequencing will be reduced, for example, by generating fewer polyclonal clusters that are not even usable for sequencing, and that the quality of sequencing data will be increased, for example, by generating fewer polyclonal clusters that may be sufficiently monoclonal for sequencing but nonetheless are contaminated by other sequences which reduce the quality of the sequencing read.

Optionally, the capture and amplification may be performed in solution, and the particles (with amplicons of the polynucleotide attached) subsequently may be disposed on a flowcell for use in sequencing the amplicons, e.g., using sequencing-by-synthesis. Because the capture and amplification are performed in solution rather than at the surface of the flowcell, the construction of the flowcell may be significantly simplified relative to that of a flowcell that is used to perform capture and amplification; for example, a flowcell for use with the present particles need not necessarily include patterned regions of capture primers, as may be the case for flowcells which are used to perform the capture and amplification. In addition to using the apertures to improve monoclonality of the clusters, monoclonality further may be improved by performing seeding and clustering using particles in solution, for example because the particles may be expected to be sufficiently spaced apart from one another in the solution that a cluster of amplicons coupled to a given particle may not be expected to be able to be contaminated by amplicons coupled to another such particle. In comparison, performing seeding and clustering on a flowcell surface runs the risk that an amplicon from one cluster may contaminate another cluster, resulting in generation of a polyclonal cluster which may not be usable for sequencing. Additionally, the present particles may increase the speed with which sequencing may be performed, for example because the capture and amplification operations may be performed off-instrument without the need to utilize time on the instrument to perform such operations; in comparison, performing capture and amplification on the flowcell can utilize significant instrument time that otherwise could have been used for sequencing.

First, some terms used herein will be briefly explained. Then, some example devices and example methods for using an aperture to capture a polynucleotide on a particle will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another. Polynucleotides that are “partially” hybridized to one another means that they have sequences that are complementary to one another, but such sequences are hybridized with one another along only a portion of their lengths to form a partial duplex. Polynucleotides with an “inability” to hybridize include those which are physically separated from one another such that an insufficient number of their bases may contact one another in a manner so as to hybridize with one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to a substrate and may hybridize to an adapter of the target polynucleotide. In some cases, a capture primer that is coupled to the substrate and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the substrate, but that may not be used to grow a complementary strand during an amplification process, may in some cases be referred to as a “seeding primer.” A capture primer that may be used to grow a complementary strand during an amplification process may in some cases be referred to as an “amplification primer.”

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible.

In some examples, a substrate is or includes a particle. As used herein, a “particle” is a small localized object which exists as a discrete unit in a given medium. In detail, the term refers to microscopic particles with sizes ranging from atoms to molecules, such as nanoparticle or colloidal particle. A particle may refer to a substrate that is sufficiently small that it may be located within a fluid (such as a liquid), that is, substantially surrounded on all sides by the fluid. As such, a particle may be carried by (moved by) the fluid in which it is located, for example by flowing the fluid from one location to another location. As explained elsewhere herein, particles can have a variety of shapes. When a particle is at least partially spherical, it may be referred to as a “bead.” In some examples, a first substrate (such as a flat or patterned surface within a flowcell) may be used to support a second substrate (such as a particle). In some examples, a particle may be porous. In some examples, a particle may be formed of multiple sub-particles.

In some examples, a substrate includes a patterned surface. A “patterned surface” refers to an arrangement of different regions in or on an exposed layer of a substrate. For example, one or more of the regions may include feature(s) that can be used to attract and/or be coupled to a particle. The feature(s) can be separated by interstitial regions where such features are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface, into which particles respectively may be disposed. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM, which is a type of polyacrylamide). The process creates hydrogel regions used to attract particles for sequencing that may be stable over sequencing runs with a large number of cycles. In some examples, covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA, which is another type of polyacrylamide) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells. The azido groups of the hydrogel material may be converted to, or coupled to, groups with a positive charge. A solution including the present particles may then be contacted with the polished substrate such that individual target particles may become attracted to, and disposed within, individual wells via interactions with the positively charged groups attached to the hydrogel material; however, the particles substantially will not occupy the interstitial regions due to absence or inactivity of the hydrogel material. Before or after contacting the particles with the substrate, the particles may be used to capture and amplify a plurality of target polynucleotides (e.g., a fragmented human genome or portion thereof). Amplicons of the target polynucleotides may be confined to the wells because the particles are confined to the wells. The amplicons then may be sequenced. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flowcell or is located in or coupled to a flowcell. Flowcells may include a flow chamber that is divided into a plurality of lanes or a plurality of sectors. Example flowcells and substrates for manufacture of flowcells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate. A hydrogel may be located on a substrate, such as on a particle or on a region of a flowcell.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate is intended to mean that the layer covers the substrate's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides coupled to respective particles) that are at different regions of an array may be differentiated from each other according to the locations of the regions in the array. An individual region of an array may include one or more molecules of a particular type. For example, a substrate region may include a single particle including amplicons of a target polynucleotide having a particular sequence. The regions of an array respectively may include different features than one another on the same substrate. Exemplary features include without limitation, wells in a substrate, particles (such as beads) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or hydrogel. Exemplary arrays in which separate substrates are located on a surface include, without limitation, those having particles (such as beads) in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A “partially” double stranded polynucleotide may have at least about 10%, at least about 25%, at least about 50%, at least about 60%, at least about 70%, at least about 80%, at least about 90% or at least about 95% of its nucleotides, but fewer than all of its nucleotides, hydrogen bonded to nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to mean a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region that includes substantially only amplicons of a given polynucleotide may be referred to as “monoclonal,” while a substrate region that includes amplicons of polynucleotides having different sequences than one another may be referred to as “polyclonal.” A substrate region that includes a sufficient number of amplicons of a given polynucleotide to be used to sequence that polynucleotide maybe referred to as “functionally monoclonal.” Illustratively a substrate region in which about 60% or greater of the amplicons are of a given polynucleotide may be considered to be “functionally monoclonal.” Additionally, or alternatively, a substrate region from which about 60% or more of a signal is from amplicons of a given polynucleotide may be considered to be “functionally monoclonal.” A polyclonal region of a substrate may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide. The action by which a polynucleotide in solution becomes coupled to a substrate may be referred to as “capturing” the polynucleotide.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a barrier (such as membrane), or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NalP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, sec Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105:20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, sec PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state barrier, e.g., a barrier including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” is intended to mean a structure that normally inhibits passage of molecules from one side of the barrier to the other side of the barrier. The molecules for which passage is inhibited can include, for example, ions and water-soluble molecules such as nucleotides or amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include membranes of biological origin, such as lipid bilayers, and non-biological barriers such as solid-state barriers or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, “synthetic” refers to a barrier material that is not of biological origin (e.g., polymeric materials, synthetic phospholipids, solid-state barriers, or combinations thereof).

As used herein, a “polymeric membrane,” “polymeric barrier,” “polymer barrier,” or a “polymer membrane” refers to a synthetic barrier that primarily is composed of a polymer that is not of biological origin. In some examples, a polymeric membrane consists essentially of a polymer that is not of biological origin. A block copolymer is an example of a polymer that is not of biological origin and that may be included in the present barriers. A hydrophobic polymer with ionic end groups is another example of a polymer that is not of biological origin and that may be included in the present barriers. Because the present barriers relate to polymers that are not of biological origin, the terms “polymeric membrane,” “polymer membrane,” “polymeric barrier,” “polymer barrier,” “membrane,” and “barrier” may be used interchangeably herein when referring to the present barriers, even though the terms “barrier” and “membrane” generally may encompass other types of materials as well.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. The first portion optionally may include an end group with a hydrophilicity that is different than that of the first type of monomer, or the second portion optionally may include an end group with a hydrophilicity that is different than that of the second type of monomer, or the first portion optionally may include an end group with a hydrophilicity that is different than that of the first type of monomer and the second portion optionally may include an end group with a hydrophilicity that is different than that of the second type of monomer. The end groups of any hydrophilic blocks may be located at an outer surface of a barrier formed using such hydrophilic blocks. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer as one another, and the second block may include a different type of monomer. In some examples, the first block may be hydrophobic, the second block may be hydrophilic, and the third block may be hydrophobic and includes the same type of monomer as the first block, in which case the triblock copolymer may be referred to as a “BAB” copolymer where “A” refers to the hydrophilic block and “B” refers to the hydrophobic blocks. In other examples, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric barrier may depend, among other things, on the respective block lengths, the type(s) of monomers used in the different blocks, the relative hydrophilicities and hydrophobicities of the blocks, the composition of the fluid(s) within which the barrier is formed, and/or the density of the polymeric chains within the barrier. During formation of the barrier, these and other factors generate forces between molecules of the polymeric chains which laterally position and reorient the molecules in such a manner as to substantially minimize the free energy of the barrier. The barrier may be considered to be substantially “stable” once the polymeric chains have completed these rearrangements, even though the molecules may retain some fluidity of movement within the barrier.

An “A-B interface” of a block copolymer (such as a diblock or triblock copolymer) refers to the interface at which the hydrophilic block is coupled to the hydrophobic block.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered to be “amphiphilic.” Illustratively, AB copolymers, ABA copolymers, and BAB copolymers all may be considered to be amphiphilic. Additionally, molecules including a hydrophobic polymer coupled to ionic end groups may be considered to be amphiphilic.

As used herein, a “solution” is intended to refer to a homogeneous mixture including two or more substances. In such a mixture, a solute is a substance which is uniformly dissolved in another substance referred to as a solvent. A solution may include a single solute, or may include a plurality of solutes. Additionally, or alternatively, a solution may include a single solvent, or may include a plurality of solvents. An “aqueous solution” refers to a solution in which the solvent is, or includes, water.

A first liquid that forms a homogeneous mixture with a second liquid is referred to herein as being “miscible” or “soluble” with the second liquid.

### Devices and Methods for Using Apertures to Capture, and Optionally Amplify, Polynucleotides on Particles

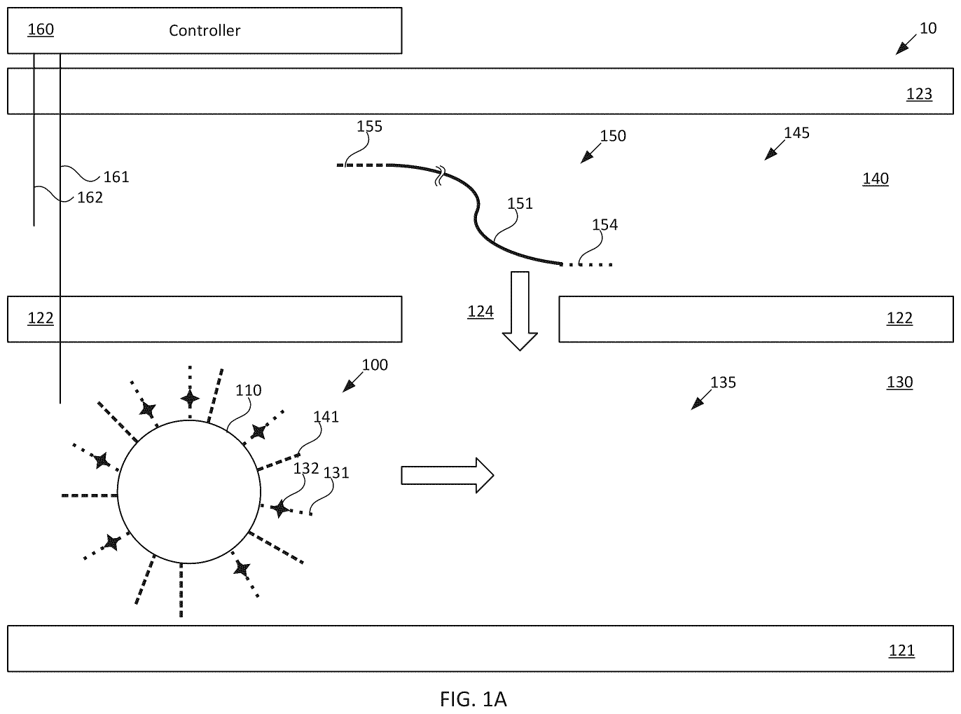

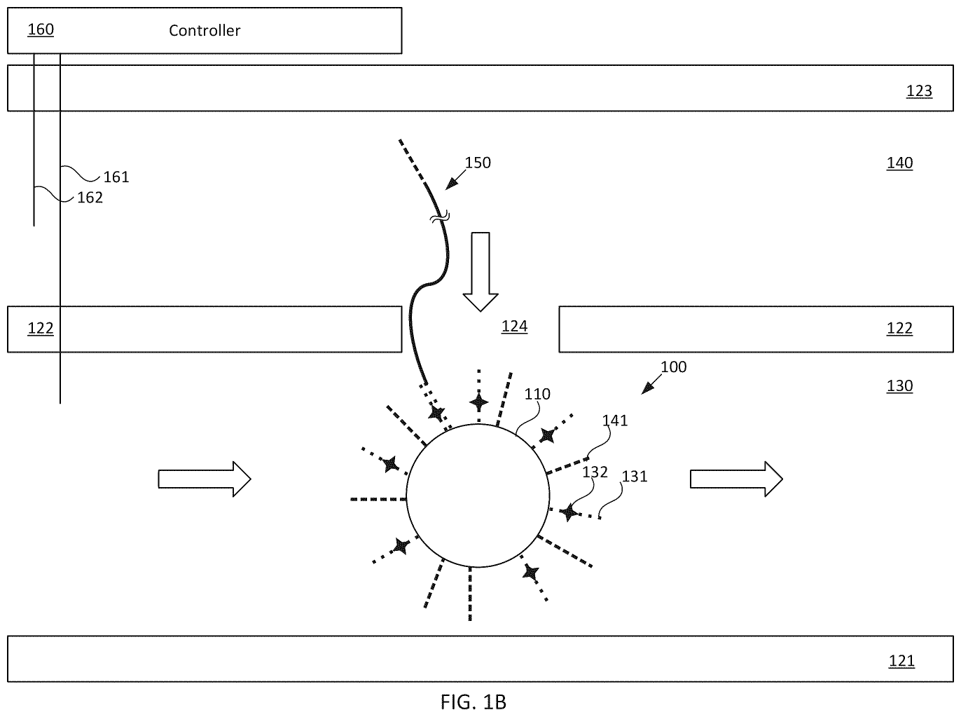

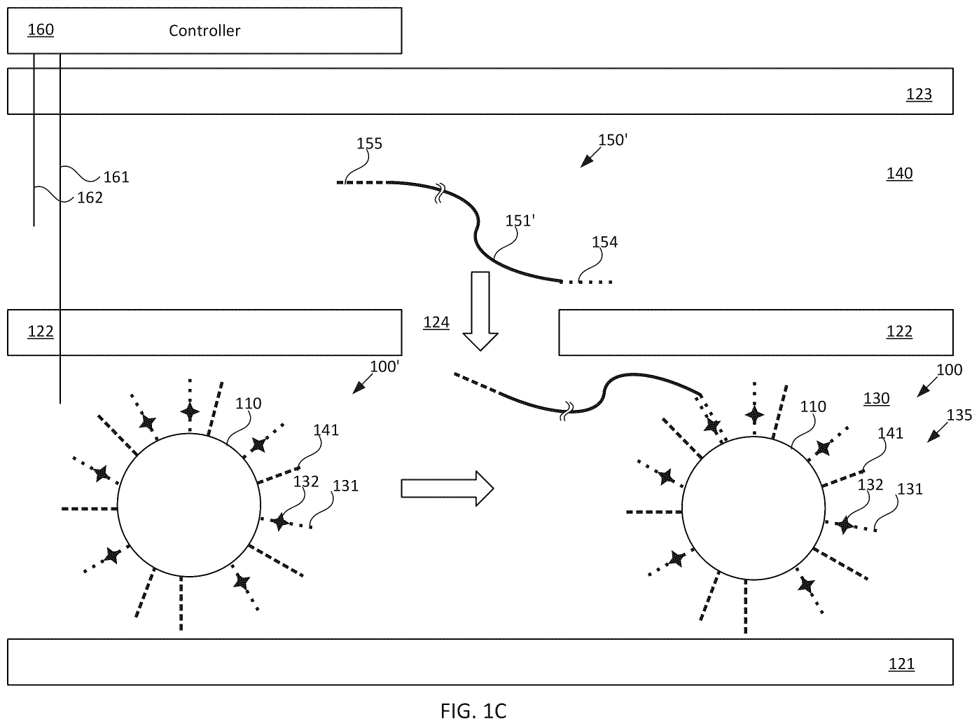

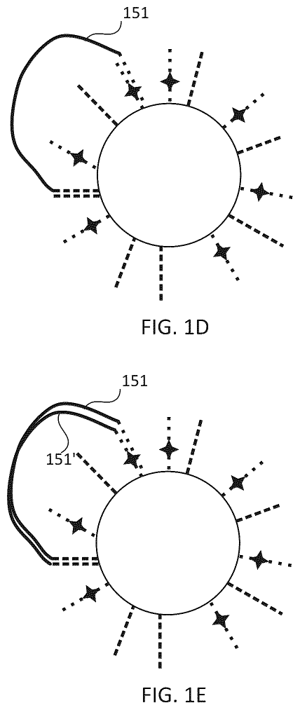

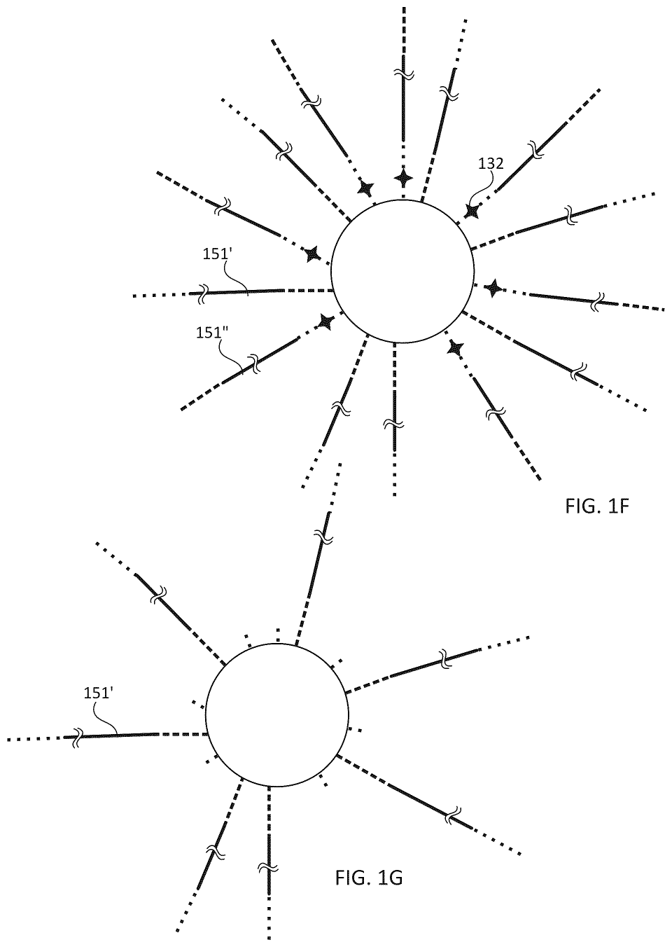

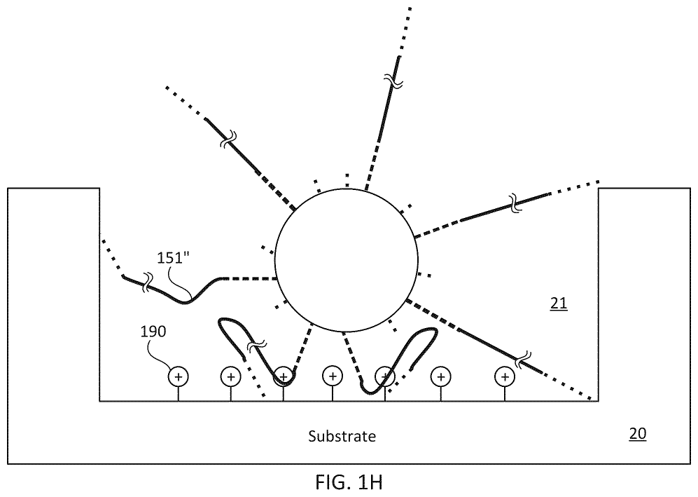

As provided herein, apertures may be used to capture, and optionally amplify, polynucleotides on particles. For example, FIGS. 1A-1H schematically illustrate an example device and operations for using an aperture to capture a polynucleotide on a particle, and optionally also amplifying the captured polynucleotide using the particle.

Referring now to FIG. 1A, device 10 illustrated in FIG. 1A includes first fluidic compartment 130, second fluidic compartment 140, and first aperture 124 defined through wall 122 between the first fluidic compartment and the second fluidic compartment 140. In the nonlimiting example illustrated in FIG. 1A, first fluidic compartment 130 is defined using at least wall 121 and wall 122, and second fluidic compartment 140 is defined using at least wall 122 and wall 123. However, it will be appreciated that fluidic compartments may have many suitable configurations, and may be formed of any suitable materials. In some examples, walls 121, 122, and 123 are substrates, some example materials for which are exemplified elsewhere herein. At the particular time illustrated in FIG. 1A, particle 100 is suspended within first fluid 135 within first fluidic compartment 130, and single-stranded polynucleotide 150 is suspended within second fluid 145 within second fluidic compartment 140. First fluid 135 and second fluid 145 may include aqueous solutions (e.g., buffer solutions). The first fluid 135 and/or second fluid 145 optionally may form droplets within an immiscible fluid that otherwise substantially fills the respective fluidic compartments. Alternatively, the first fluid 135 may substantially fill the first fluidic compartment 130 and/or the second fluid 145 may substantially fill the second fluidic compartment.

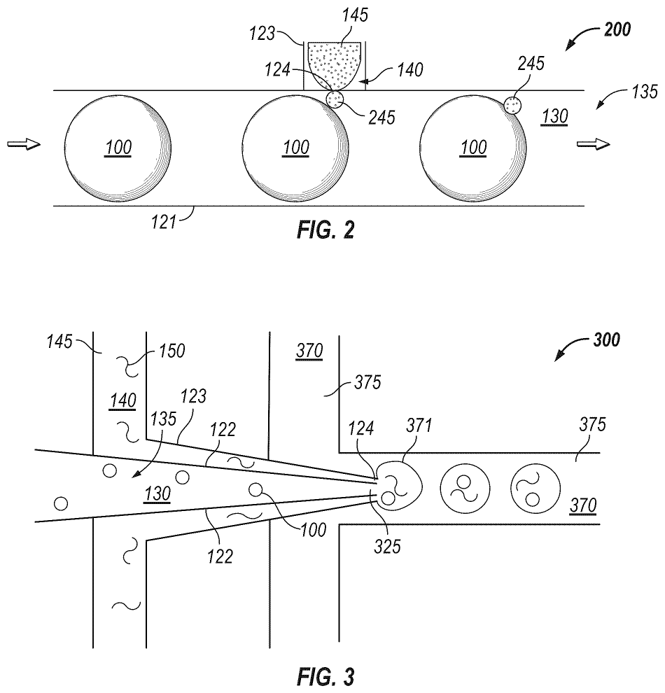

As illustrated in FIG. 1A, device 10 also may include controller 160 to transport a particle within the first fluidic compartment 130 to the first aperture 124, and to transport a polynucleotide from the second fluidic compartment 140 to the first fluidic compartment through the first aperture so as to hybridize the polynucleotide to the capture primer. As illustrated in FIG. 1A, controller 160 may be configured to control the motion of particle(s) within the first fluidic compartment 130 and/or to control the motion of polynucleotide(s) within second fluidic compartment 140 in such a manner that the particle captures substantially a single polynucleotide. Such motion is intended to be illustrated in FIGS. 1A-1C by respective block arrows. Illustratively, controller 160 may control the flow of first fluid 135, and/or may control the motion of particle 100 within first fluid 135, via first communication pathway(s) 161 with any suitable device or combination of devices, such as a mechanical pump, digital fluidics, ultrasonics, heat, electric pulse, and/or shape changing material. Similarly, controller 160 may control the flow of second fluid 145, and/or may control the motion of polynucleotide 150 within second fluid 145, via second communication pathway(s) 162 with any suitable device or combination of devices, such as a mechanical pump, digital fluidics, ultrasonics, heat, electric pulse, and/or shape changing material. Optionally, controller 160 may use such device(s) to transport particle 100 to the first aperture 124, and may use such device(s) to temporarily hold particle 100 at the first aperture for a sufficient amount of time to capture polynucleotide 150 on particle 100. In some examples such as explained further below with reference to FIGS. 2-9, controller 160 may control the transport of particle 100 and the transport of polynucleotide 150 in the same manner as one another, or may control the transport of particle 100 and the transport of polynucleotide 150 in different manners than one another.

Referring now to FIG. 1B, controller 160 controls the transport of particle 100 and/or polynucleotide 150 such that polynucleotide 150 is transported through aperture 124 at a time when particle 100 is sufficiently close to the aperture to capture polynucleotide 150. For example, particle 100 may include a capture primer, and polynucleotide 150 may include an adapter that is complementary to the capture primer. As such, when controller 160 brings particle 100 and polynucleotide 150 into sufficient proximity to one another, the adapter may hybridize to the primer, and as such the particle may be considered to capture the polynucleotide in a manner such as illustrated in FIG. 1B. For example, particle 100 may include a substrate 110 to which is coupled plurality of each of first and second amplification primers 131, 141 which have orthogonal sequences to one another. In one nonlimiting example, capture primers 131 may include P5, and capture primers 141 may include P7. As illustrated in FIG. 1A, polynucleotide 150 may include adapter 154 which is complementary to capture primers 131, and adapter 155 which is complementary to capture primers 141. As illustrated in FIG. 1B, adapter 154 hybridizes to capture primer 131, and as such particle 110 captures polynucleotide 150. Optionally, the particle also may be used to amplify the polynucleotide in a manner such as will be described with reference to FIGS. 1D-1G, and the resulting substantially monoclonal cluster of amplicons may be disposed in a flowcell and sequenced in a manner such as will be described with reference to FIG. 1H.

Although FIGS. 1A-1B may focus on the manner in which device 10 uses aperture 124 to control a single particle's capture of a single polynucleotide, it will be appreciated that device 10 may be used to capture a plurality of polynucleotides on respective particles in relatively rapid succession, so as to prepare substantially monoclonal clusters coupled to respective particles. Indeed, although only one particle 100 is illustrated in FIGS. 1A-1B, it will be appreciated that fluid 130 may include thousands, or even millions, of particles that have substantially the same configuration as one another. Additionally, although only one polynucleotide 150 is illustrated in FIGS. 1A-1B, it will be appreciated that fluid 145 may include thousands, or even millions, of polynucleotides which in some cases may have different lengths than one another. Controller 160 and aperture 124 may be used to inhibit contact between these additional polynucleotides and additional particles other than in a controlled manner such as described with reference to FIGS. 1A-1B. For example, as illustrated in FIG. 1C, fluid 135 further may include a plurality of additional particles 100, for simplicity illustrated as a second particle denoted 100′ which is configured similarly as particle 100. Additionally, fluid 145 further may include a plurality of additional polynucleotides 150, for simplicity illustrated as second polynucleotide 150′ which is configured similarly as particle 100, but which includes a different polynucleotide 151′ to be captured, amplified, and sequenced.

In addition to controlling the movement of particle 100, controller 160 may control the movement of additional ones of the particles. Illustratively, controller 160 may move fluid 135 within first fluidic compartment 130 so as to sequentially transport different particles past aperture 124. For example, as illustrated in FIG. 1C, motion of fluid 135 may carry particle 100 away from aperture 124 and may carry particle 100′ toward aperture 124. Before controller 160 transports particle 100 to aperture 124 and after the controller moves particle 100 away from aperture 124 after the polynucleotide is hybridized to that particle's capture primer, wall 122 may inhibit the particle from capturing any other polynucleotides besides the one polynucleotide 150 illustrated in FIG. 1B. Similarly, before controller 160 respectively transports each of the other particles (illustratively, particle 100′) to aperture 124 and after the controller moves such particles away from aperture 124, after the respective polynucleotide is hybridized to that particle's capture primer, wall 122 may inhibit the particles from capturing any other polynucleotides besides that polynucleotide in a manner such as illustrated in FIG. 1B. Additionally, controller 160 may move fluid 145 within second fluidic compartment 140, or may move polynucleotides 150 within fluid 145, so as to sequentially transport different polynucleotides through aperture 124 at times at which respective particles are at aperture 124. For example, as illustrated in FIG. 1C, controller 160 may transport polynucleotide 150′ through aperture 124 at a time when particle 100′ is at aperture 124, so that particle 100′ may capture polynucleotide 150′ in a manner similar to that described with reference to FIG. 1A. Before controller 160 transports particle 100 to aperture 124 and before controller 160 transports polynucleotide 150 through aperture 124, and after the controller moves particle 100 away from aperture 124, wall 122 may inhibit the particle from capturing any other polynucleotides besides the one polynucleotide 150 illustrated in FIG. 1B. Similarly, before controller 160 respectively moves each of the other particles (illustratively, particle 100′) to aperture 124 and before controller 160 transports other respective polynucleotides (illustratively, polynucleotide 150′) through aperture 124, and after the controller moves such particles away from aperture 124, wall 122 may inhibit the particles from capturing any other polynucleotides besides the one respective polynucleotide (illustratively polynucleotide 150′) in a manner such as illustrated in FIG. 1C. Accordingly, aperture 124 is used to capture substantially one polynucleotide per particle. Such polynucleotide then may be amplified to generate a substantially monoclonal cluster, which subsequently may be sequenced.

Referring now to FIG. 1D, adapter 154 of captured polynucleotide 150 may hybridize to a capture primer 131 of particle 100 to form a first duplex, and adapter 155 of captured polynucleotide 150 may hybridize to a capture primer 141 of particle 100 to form a second duplex. Polynucleotide 150 then may be amplified, e.g., using a bridge amplification or other suitable amplification operations such as described elsewhere herein or otherwise known in the art. For example, as illustrated in FIG. 1E, a polymerase may be used to extend primer 131 based on the sequence of polynucleotide 150 to generate a first amplicon 151′ of polynucleotide 150 that is covalently coupled to particle 100. Amplicon 151′ repeatedly may be further amplified using similar operations. For example, it may be seen that the composition of FIG. 1F includes amplicon 151′ and a plurality of additional amplicons 151″ of amplicon 151′ that are formed using a mixture of capture primers 131 and 141 for the amplification.

Amplification operations may be formed any suitable number of times so as to substantially fill the particle with a substantially monoclonal cluster, e.g., with amplicons of target polynucleotide 150. For example, the amplicons coupled to the particle may include at least about 90% amplicons of one selected target polynucleotide, or at least about 95% amplicons of one selected target polynucleotide, or at least about 98% amplicons of one selected target polynucleotide, or at least about 99% amplicons of one selected target polynucleotide, or about 100% amplicons of one selected target polynucleotide. If amplification operations are repeated until the particle is substantially full, both adapters of the resulting amplicons may not necessarily be hybridized to corresponding capture primers or orthogonal capture primers, and as such the amplicons may extend linearly away from the particle 100 as illustrated in FIG. 1F. Although FIGS. 1A-1F illustrate an example in which polynucleotide capture and amplification operations are performed in solution, it should be appreciated that in other examples, any suitable ones of such operations instead may be performed after disposing the particle on a substrate, e.g., within recess 21 in a manner such as will be described with reference to FIG. 1H.

In some examples, certain capture primers and orthogonal capture primers may include non-nucleotide moieties. Such non-nucleotide moieties may include, but are not limited to, excision moieties via which a portion of the capture primers selectively may be removed. For example, capture primers 131 optionally may include excision moieties 132 and/or capture primers 141 optionally may include excision moieties (excision moieties of capture primers 141 not specifically shown in FIGS. 1A-1F). The excision moieties 132 of capture primers 131 may be of the same type as, or a different type than, the excision moieties of capture primers 141. The excision moieties may be located at any suitable position along the length of any suitable primer(s) and may be, but need not necessarily be, the same type of excision moiety as one another. Following a desired number of amplification operations such as described with reference to FIGS. 1D-1F, portions of capture primers 131 may be removed by reacting a suitable enzyme or reagent with the respective excision moieties, and/or portions of capture primers 141 may be removed by reacting a suitable enzyme or reagent with the respective excision moieties. The enzyme or reagent used with the excision moieties of capture primers 131 may be the same as, or different than, the enzyme or reagent used with excision moieties of capture primers 141. Excision moieties 132 may be used to remove amplicons that are oriented in a selected direction, so that the remaining amplicons are oriented substantially in the same direction as one another in a manner such as illustrated in FIG. 1G. In some examples, device 100 may be configured to perform one or more amplification operations such as described with reference to FIGS. 1D-1G, while in other examples such operations may be performed separately from device 100.

In some examples, following the seeding and amplification operations, particle 100 with amplicons 151′ and 151″ coupled thereto may be disposed within a flowcell and the amplicons sequenced, e.g., using sequencing-by-synthesis. Illustratively, as shown in FIG. 1H, a substrate 20 (e.g., a surface of a flowcell) may include a plurality of positively charged moieties 190. In the nonlimiting example illustrated in FIG. 1H, the substrate 20 includes a recess 21 in which the positively charged moieties 190 are disposed. Optionally, the positively charged moieties may be coupled to a hydrogel (not specifically illustrated) which is located on the substrate (e.g., within the recess 21). The amplicons 151′, 151″ may have a negative charge that is electrostatically attracted to the positively charged moieties 190. This electrostatic attraction may draw particle 100 and/or amplicons 151′, 151″ into contact with the substrate. Additionally, this electrostatic attraction and/or other force(s) may retain particle 100 and/or amplicons in contact with the substrate 20. The amplicons then may be sequenced e.g., using sequencing-by-synthesis operations that use reagents which may be flowed across the substrate 10. For example, sequencing-by-synthesis operations (not specifically illustrated) may generate, and determine the sequence of, amplicons 151′ and 151″ as illustrated in FIG. 1H.

As noted further above, a wide variety of fluidic control devices and configurations may be used to capture polynucleotides on particles. Some such examples will be described with reference to FIGS. 2-9, but it will be appreciated that such examples are purely illustrative, and not intended to be limiting.

In some examples, controller 160 is to transport the polynucleotide through the first aperture before the polynucleotide is hybridized to the capture primer. For example, in a manner such as illustrated in FIG. 1A, before being transported through the first aperture 124, the polynucleotide 150 is located entirely in the second fluidic compartment. FIG. 2 illustrates a nonlimiting example of a configuration in which the controller is to transport the polynucleotide through the first aperture by flowing a fluid, in which the polynucleotide is suspended, through the aperture and into the first fluidic compartment. Device 200 illustrated in FIG. 2 includes first fluidic compartment 130 defined using substrate walls 121 and 122, and second fluidic compartment 140 defined using substrate walls 122 and 123 in a manner similar to that described with reference to FIGS. 1A-1H. First fluidic compartment 130 and second fluidic compartment 140 may be arranged in a T-junction microfluidic device, in which polynucleotides 150 within second fluidic compartment 140 may be transported from second fluidic compartment 140 to first fluidic compartment 130 through aperture 124 within wall 122. More specifically, controller 160 (not specifically illustrated in FIG. 2) flows fluid 135 through first fluidic compartment 130 so as to sequentially transport particles 100 past aperture 124 at a controlled rate. Optionally, and in a manner such as described above, in addition to or as an alternative to controlling the flow of fluid 135, controller 160 may use a device to control the transport of particles 100 within fluid using ultrasonics, heat, electric pulse, and/or shape changing material. Additionally, controller 160 flows fluid 145 through second fluidic compartment 140 so as to sequentially emit droplets 245 of fluid 145 through aperture 124 at a rate which is appropriately synchronized to the rate of flow of fluid 135, such that approximately one droplet 245 is emitted when a corresponding particle is located at aperture 124 in a manner such as illustrated in FIG. 2.

By dispensing droplets 245 directly onto respective particles 100, and suitably sizing the first fluidic compartment 130 and particles 100, diffusion and laminar flow may be substantially inhibited that otherwise may cause droplets 245 to dissociate from particles 100. In some examples, the internal dimension(s) of the first fluidic compartment 130 and the external dimension(s) of particles 100 are similar to one another, e.g., as shown in FIG. 2, while the length of the first fluidic compartment may be significantly longer than shown in FIG. 2. For example, the particles may have external dimension(s) of at least about 70% of the respective internal dimension(s) of the first fluidic compartment, or at least about 80% of the respective internal dimension(s) of the first fluidic compartment, or at least about 90% of the respective internal dimension(s) of the first fluidic compartment, or at least about 95% of the respective internal dimension(s) of the first fluidic compartment. Illustratively, the particles may have external dimension(s) of about 50 nm to about 5000 nm, e.g., about 50 nm to about 1000 nm. Additionally, or alternatively, the first fluidic compartment may have internal dimension(s) which are similar to that of the particles, e.g., about 50 nm to about 5000 nm, e.g., about 50 nm to about 1000 nm. In some examples, the particles may be about 70% to about 95% the size of the internal dimension(s) of the first fluidic compartment, e.g., about 80% to about 90% the size of the internal dimension(s) of the first fluidic compartment, so as to readily be transported through the first fluidic compartment.

In some examples, the size of droplets 245 and the concentration of polynucleotides 150 within fluid 135 is selected such that at least some of droplets 245 contain either one polynucleotide 150 or no polynucleotides 150. For example, the plurality of polynucleotides 150 may be suspended in the fluid at a concentration of less than about a pM, or between about 0.001 mM and about 100 mM, or between about 0.01 mM and about 50 mM. or between about 0.1 mM and about 25 mM, or between about 1 nM and about 100 nM. Additionally, or alternatively, droplets 245 may have a size of about 50 nm in diameter to about 5000 nm in diameter. For an example droplet (245) of 50 nm in diameter, the concentration of polynucleotides to obtain a single polynucleotide molecule inside this volume is about 25 mM. For an example droplet (245) of 5000 nm in diameter, the concentration of polynucleotides to obtain a single polynucleotide molecule inside this volume is about 0.15 mM. Before being disposed in respective droplets, the polynucleotides may be distributed throughout the fluid in any suitable manner, such as with a mixing device (illustratively using sonication or stirring). At a suitable droplet size and concentration of polynucleotides, based on the Poisson statistical distribution, if the concentration of the nucleotides in the feeding solution 145, is set such as the droplet 245 contains one molecule in average, about 37% of the droplets may be expected to be empty, about 37% of the droplets may be expected to contain exactly one polynucleotide, about 18% of the droplets may be expected to contain exactly two polynucleotides, and the remaining 8% of the droplets may be expected to contain three or more polynucleotides. By using an even lower concentration of polynucleotides, a sub-Poisson distribution of polynucleotides in the droplets may be obtained, for example such that approximately 80% of the particles 100 may be expected not to capture any polynucleotide, approximately 20% of the particles 100 may be expected to capture exactly one polynucleotide, and a negligible percentage of the particles 100 may be expected to capture two or more polynucleotides; or such that approximately 90% of the particles 100 may be expected not to capture any polynucleotide, approximately 10% of the particles 100 may be expected to capture exactly one polynucleotide; and a negligible percentage of the particles may be expected to capture two or more polynucleotides.

Particles which capture a polynucleotide may be separated from particles which do not capture a polynucleotide using any suitable operation(s) which device 200 may be configured to perform, or which may be performed separately from device 200. For example, the collection of particles (each of which may or may not have captured a polynucleotide) may be subjected to amplification operations such as described with reference to FIGS. 1D-1G. A cluster of amplicons is generated on particles which captured a polynucleotide, while a cluster of amplicons is not generated on particles which did not capture a polynucleotide. The particles with a cluster of amplicons have a different size, and a different charge, than particles without a cluster of amplicons. As such, the particles with the cluster of amplicons may be separated via enrichment based on size and/or charge from the particles without a cluster of amplicons. Controller 160 may be configured to perform such separation, or the separation may be performed using a different device and/or operations. Optionally, separated particles without a cluster of amplicons may be recycled back into the first fluidic compartment 135 to be contacted with another droplet 245, for example using a fluidic channel between fluidic compartment 135 and a fluidic compartment at which the separation is performed. Such recycling may significantly reduce waste of otherwise unused particles. Additionally, although the likelihood of any given particle capturing a polynucleotide on a single given trip past aperture 124 may be relatively low (e.g., about 20% or less), device 100 may transport that particle past aperture 124 repeatedly until that particle captures a polynucleotide. As such, the likelihood of a given particle eventually capturing a polynucleotide may be relatively high, and even may approach 100%.

FIG. 3 illustrates another example in which controller 160 is to transport the polynucleotide through the first aperture before the polynucleotide is hybridized to the capture primer. FIG. 3 illustrates a nonlimiting example of a configuration in which the controller is to transport the polynucleotide through the first aperture 124 by flowing a fluid, in which the polynucleotide is suspended, through the aperture and into a third fluidic compartment into which the particle also is flowed. More specifically, device 300 illustrated in FIG. 3 includes first fluidic compartment 130 defined using substrate wall(s) 122 and 123, and second fluidic compartment defined using substrate walls 121 and 122 in a manner similar to that described with reference to FIGS. 1A-1H. Device 300 also includes third fluidic compartment 370 which is fluidically coupled to second fluidic compartment 140 via first aperture 124, and fluidically coupled to first fluidic compartment 130 via second aperture 325. First fluidic compartment 130, second fluidic compartment 140, and third fluidic compartment 370 may be arranged in a double-T-junction microfluidic device, in which polynucleotides 150 within second fluidic compartment 140 may be transported from second fluidic compartment 140 to third fluidic compartment 370 through aperture 124. Although second fluidic compartment 140 and third fluidic compartment 370 are illustrated as having a T configuration having two branches flowing towards the first fluidic compartment 130, compartments 140 and 370 instead may be configured to have a single branch.

More specifically, under the control of controller 160 (controller not specifically illustrated in FIG. 3), fluid 145 containing polynucleotides 150 is flowed from the second fluidic compartment 140 to the third fluidic compartment 370 via first aperture 124. Additionally, under the control of controller 160, fluid 135 containing particles 100 is flowed from the first fluidic compartment 130 to the third fluidic compartment 370 via second aperture 325. First fluid 135 and second fluid 145 may be miscible with one another (e.g., may both be aqueous), and may be immiscible with third fluid 375 within third fluidic compartment 370 (e.g., fluid 375 may be an oil). Controller 160 may flow fluid 145 at a rate which is appropriately synchronized to the rate of flow of fluid 135, such that approximately one droplet of fluid 145 is emitted from aperture 124 at a time when a corresponding particle is located at aperture 325 in a manner such as illustrated in FIG. 3. Droplets of first fluid 135 and second fluid 145 may combine together to form discrete droplets 371 which are suspended in, and substantially surrounded by, fluid 375. Similarly as the transport of particles 100 using fluid 135 described with reference to FIGS. 1A-1H, controller 160 may transport droplets 371 using the flow of fluid 375. Note that use of the terms “first,” “second,” and “third” herein is arbitrary. Illustratively, when polynucleotides 150 are transported through aperture 124 into the fluidic compartment within which particles 100 are located, such fluidic compartment equivalently may be referred to as a first, second, or third fluidic compartment.

In a manner similar to that described with reference to FIG. 2, the concentration of polynucleotides 150 in fluid 145 may be selected such that the droplets of fluid 145 contain either one polynucleotide or zero polynucleotides, illustratively in the range of about 1 nM to about 100 nM, or other ranges such as described with reference to FIGS. 1A-1H. Similarly, the concentration of particles in fluid 135 may be selected such that the droplets of fluid 135 contain either one particle or zero particles, illustratively in the range of about 1 nM to about 100 nM. As such, some of droplets 371 may include exactly one particle and exactly one polynucleotide, and within these droplets the particle captures (and optionally amplifies) the polynucleotide in a manner similar to that described with reference to FIGS. 1A-1H. In other droplets that lack a particle and/or lack a polynucleotide, no capture or amplification may be performed. In some examples, the concentration of particles in fluid 135 may be somewhat higher than the concentration of polynucleotides in fluid 145, such that at least some droplets contain two or more particles. Each particle in the droplet potentially may amplify the same nucleotide to generate duplicate monoclonal clusters, but such duplication is not expected to be problematic or to occur particularly often. Particles that captured and amplified a polynucleotide may be separated from particles that did not capture and did not amplify a polynucleotide in a manner similar to that described with reference to FIG. 2, and the latter type of particles optionally may be recycled in a manner such as described with reference to FIG. 2.

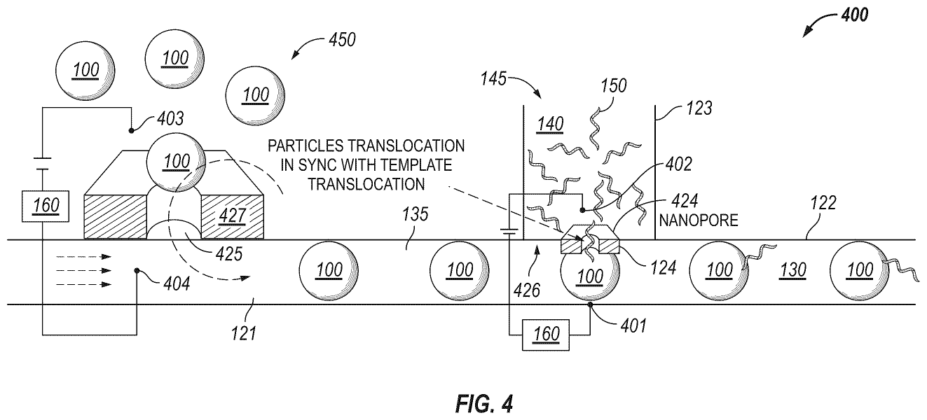

FIG. 4 illustrates another example in which controller 160 is to transport the polynucleotide through the first aperture before the polynucleotide is hybridized to the capture primer. More specifically, FIG. 4 illustrates a nonlimiting example of a configuration in which the controller is to synchronize transport of the particle to the first aperture with transport of the polynucleotide through the first aperture.

More specifically, device 400 illustrated in FIG. 4 includes first fluidic compartment 130, second fluidic compartment 140, and first aperture 124 defined through wall 122 between the first fluidic compartment 130 and the second fluidic compartment 140. In the nonlimiting example illustrated in FIG. 4, first fluidic compartment 130 is defined using at least wall 121 and wall 122, and second fluidic compartment 140 is defined using at least wall 122 and wall 123. However, it will be appreciated that fluidic compartments may many suitable configurations, and may be formed of any suitable materials. In some examples, walls 121, 122, and 123 are substrates, some example materials for which are exemplified elsewhere herein. In the example shown in FIG. 4, a portion of wall 122 may be formed using barrier 426, e.g., a lipid bilayer membrane or block copolymer membrane within which a nanopore 424 forming aperture 124 is suspended. Similarly as described with reference to FIGS. 1A-1H, particles 100 are suspended within first fluid 135 within first fluidic compartment 130, and single-stranded polynucleotides 150 are suspended within second fluid 145 within second fluidic compartment 140. First fluid 135 and second fluid 145 may include aqueous solutions (e.g., buffer solutions). In the example shown in FIG. 4, the first fluid 135 may substantially fill the first fluidic compartment 130 and/or the second fluid 145 may substantially fill the second fluidic compartment.

Similarly as described with reference to FIGS. 1A-1H, device 400 also may include controller 160 to transport a particle within the first fluidic compartment 130 to the first aperture 124, and to transport a polynucleotide from the second fluidic compartment 140 to the first fluidic compartment through the first aperture 124 so as to hybridize the polynucleotide to the capture primer. In some examples shown in FIG. 4, controller 160 may synchronize transport using operations that include detecting transport of a polynucleotide 150 through the first aperture 124. Additionally, or alternatively, controller 160 may electronically control transport of the polynucleotide through the first aperture 124 and into the first fluidic compartment 130. For example, as shown in FIG. 4, first aperture 124 may include a nanopore which is suspended in a barrier 426. Controller 160 may include a first electrode 401 in electrical communication with second fluid 145 in second fluidic compartment 140, and a second electrode 402 in electrical communication with first fluid 135 in first fluidic compartment 135 (electrodes schematically represented by black dots). Controller 160 may detect changes in an electrical characteristic of the first aperture 124 responsive to translocation of a polynucleotide through that aperture. For example, translocation of the polynucleotide 150 through aperture 124 may alter the rate at which a salt in fluid 135 and/or in fluid 145 moves through aperture 124, and thus may detectably alter the electrical conductivity of aperture 124 in such a manner as to be detected by controller 160. Additionally, or alternatively, controller 160 may apply a voltage between first electrode 401 and second electrode 402 to generate a potential that translocates a single polynucleotide 150 through aperture 124 at a specified time.

Controller 160 may transport a particle 100 to the first aperture 124 based on the transport of a polynucleotide through the first aperture, whether such transport is detected by the controller 160 and/or is electronically caused by the controller 160. More specifically, controller 160 may electronically control transport of a single particle 100 through second aperture 425 and into the first fluidic compartment 130 at a time which is appropriately synchronized to the time at which a single polynucleotide 150 is transported through aperture 124. Illustratively, controller 160 may include a third electrode 403 which is in electrical contact with a fluidic compartment 450 which is disposed on one side of nanopore 427 in which second aperture 425 is defined, and a fourth electrode 404 in electrical communication with first fluid 130 in first fluidic compartment 135. In a manner similar to that described with reference to nanopore 424, controller 160 may detect changes in an electrical characteristic of the second aperture 425 responsive to translocation of a particle through that aperture. For example, translocation of the particle 100 through aperture 425 may alter the rate at which a salt in fluid 135 moves through aperture 425, and thus may detectably alter the electrical conductivity of aperture 425 in such a manner as to be detected by controller 160. Additionally, or alternatively, controller 160 may apply a voltage between third electrode 403 and fourth electrode 403 that generates a potential that translocates a particle 100 through aperture 425 at a specified time. For example, controller 160 may synchronize the translocation of single particles 100 and/or single polynucleotides 150 through their respective apertures 425 and 124 such that one of particles 100 is sufficiently close to aperture 124 to capture one of polynucleotides 150 at a time when that polynucleotide translocates through aperture 124. In some examples, controller 160 translocates a particle 100 through aperture 425 in response to detecting translocation of a polynucleotide 150 through aperture 124. In other examples, controller 160 translocates a polynucleotide 150 through aperture 124 responsive to detecting translocation of a particle 100 through aperture 425. In still other examples, controller 160 translocates a polynucleotide 150 through aperture 124 and translocates a particle through aperture 425 at times that are synchronized to one another. In any of such examples, as part of such synchronization, controller 160 further may control the flow of first fluid 135, and/or may control the motion of particles 100 within first fluid 135, in a manner such as described with reference to FIGS. 1A-1H.

In one specific, nonlimiting example, controller 160 is configured such that every time a polynucleotide 150 translocates through aperture 124, the controller detects a change in an ionic current through that aperture. Barrier 426 having nanopore 424 therein is sandwiched between second fluidic compartment 140 and first fluidic compartment 130. Controller 160 applies a bias voltage across barrier 426, e.g., using first electrode 401 and second electrode 402. Single-stranded polynucleotides 150 (e.g., DNA templates) are disposed within second fluidic compartment 140, together with suitable fluid 145, such as an aqueous buffer (electrolyte solution). Controller 160 can electrophoretically transport the polynucleotides 150 through aperture 124. When the polynucleotides 150 pass through aperture 124, a blocked ionic current with a unique magnitude (ΔI) and/or dwell time (Δt) may be generated that controller 160 detects. Accordingly, in some examples, controller 160 may both control and detect translocation of respective polynucleotides 150 through aperture 124. In some examples, controller 160 uses feedback control over the number of particles 100 translocating through aperture 425. For example, controller 160 may be configured such that responsive to detecting that a particle 100 translocates through aperture 425 under a potential difference which controller 160 applies across the third and fourth electrodes 403, 404 (e.g., via a signature change in current intensity), controller 160 stops applying the potential difference across aperture 425 and begins applying a potential difference across aperture 124 at an appropriate time to translocate a polynucleotide 150 on top of particle 100.

In some examples, translocation of the polynucleotide 150 through the aperture 124 is inhibited at times other than under the control of controller 160. For example, a duplex DNA strand, or a biotinylated or capped version of the polynucleotide, is used, wherein the steric hindrance inhibits translocation absent appropriate stimulus by controller 160. The translocation can be favored by applying an electric pulse that strips off the duplexed DNA strand, biotinylation, or cap, allowing the single-stranded polynucleotide 150 to translocate through aperture 124. In certain examples, a voltage is always applied to circuit 160 but the circuit is open at times when particle 100 is not located at the aperture because the carrier liquid 130 is an electrical insulator (such as silicone oil); at a time when a particle 100 is located at the aperture 124, the particle closes the circuit and a polynucleotide 150 is translocated through the aperture 124.

As intended to be illustrated in FIG. 4, controller 160 times the translocation of particles 100 through aperture 425 to the translocation of polynucleotides 150 through aperture 124 in a 1:1 manner and so that a particle is at aperture 124 when a polynucleotide is translocated through that aperture. Electrode 401 may be placed opposite to aperture 124 to inhibit current flow disruptions through that aperture when a particle 100 is located at the aperture 124. Hybridization between a polynucleotide 150 and a capture primer (e.g., 131 or 141) on particle 100 may occur within a millisecond. Since the flow of polynucleotides through aperture 425 will be in the direction of the current flow between electrode 402 and electrode 401, the particle 100 may be positioned in between these two electrodes so that it is in the path of the DNA flow as it goes underneath the nanopore.

In examples such as described with reference to FIG. 4, nanopore 427 and nanopore 424 independently may include a biological nanopore or a solid-state nanopore. Apertures 425 and 124 may have different sizes than one another. For example, second aperture 425 may be sized so as to permit a single particle 100 to pass therethrough at a time. Illustratively, the particles may have external dimension(s) of about 50 nm to about 5000 nm, e.g., about 50 nm to about 1000 nm. Additionally, or alternatively, the second aperture 425 may have a diameter which is similar to that of the particles, e.g., about 50 nm to about 5000 nm, e.g., about 50 nm to about 1000 nm. In some examples, the particles 100 have a diameter which is about 80% to 95% the diameter of second aperture 425. Similarly, first aperture 124 illustrated in FIG. 4 (and in other examples herein) may be sized so as to permit a single polynucleotide 150 to pass therethrough at a time. Illustratively, first aperture 124 may have a diameter of about 2 nm to about 20 nm.

Non-limiting example of solid-state nanopores and their uses and preparation are described in the following references, the entire contents of each of which are incorporated by reference herein: Chen et al., “Fabrication and applications of solid-state nanopores,” Sensors 19(8): 1886, 29 pages (2019); Jain et al., “Integration of solid-state nanopores in microfluidic networks via transfer printing of suspended membranes,” Anal. Chem. 85(8): 3871-3878 (2013); Fu et al., “Microfluidic systems applied in solid-state nanopore sensors,” Micromachines 11(3): 332, 20 pages (2020); and Rahman et al., “On demand delivery and analysis of single molecules on a programmable nanopore-optofluidic device,” Nature Communications 10, article number: 3712, 7 pages (2019).

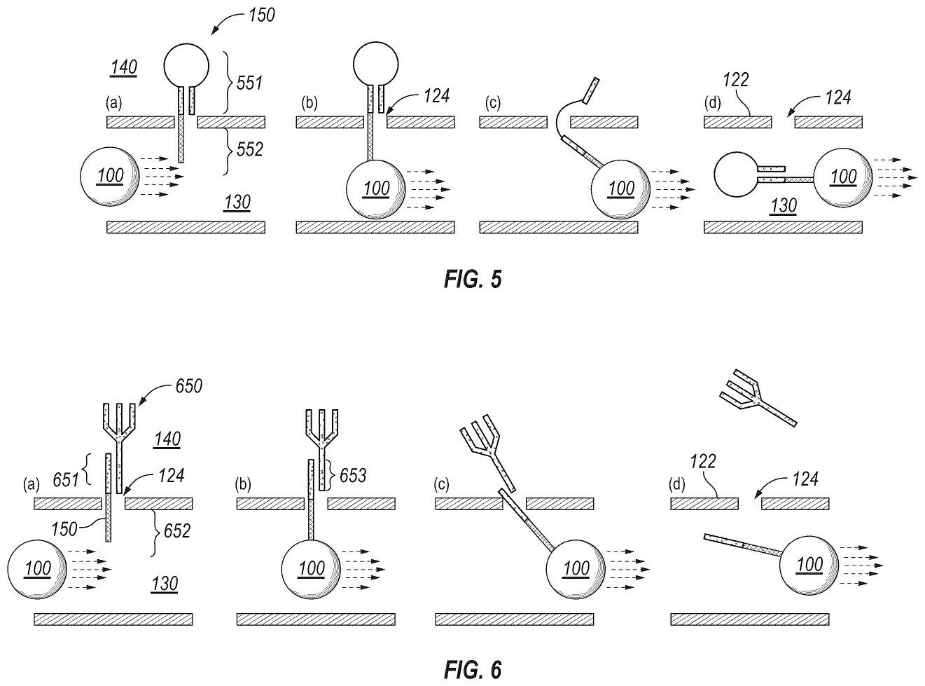

In still other examples of device 10 described with reference to FIGS. 1A-1H, polynucleotide 150 is transported through first aperture 124 in response to hybridizing the polynucleotide to the capture primer of particle 100. For example, FIG. 5 illustrates an example in which, in operation (a) prior to the polynucleotide 150 being hybridized to the capture primer of particle 100, a first portion 551 of the polynucleotide is located in the second fluidic compartment 140 and a second portion 552 of the polynucleotide is located in the first fluidic compartment 130. The first portion 551 of the polynucleotide 150 may retain the first portion of the polynucleotide in the second fluidic compartment. For example, as illustrated in FIG. 5, the first portion 551 of the polynucleotide may include a DNA loop. In operation (b) of FIG. 5, when particle 100 is located at aperture 124 (e.g., by being carried past aperture 124 via flow of a fluid within first fluidic compartment 130), particle 100 captures polynucleotide 150 in a manner such as described with reference to FIGS. 1A-1H, e.g., via hybridization between polynucleotide 150 and a capture primer on particle 100. In operation (c) of FIG. 5, responsive to the polynucleotide 150 hybridizing to the capture primer, the first portion 551 of the polynucleotide is transported from the second fluidic 140 compartment into the first fluidic compartment 130 through first aperture 124. For example, as illustrated in operation (c) of FIG. 5, when polynucleotide 150 is hybridized to the capture primer of particle 100, force from the particle dissociates the DNA loop. As illustrated in operation (d), aperture 124 is free to receive another polynucleotide 150. Additionally, the DNA loop optionally may re-form within first fluidic compartment 130. Optionally, the DNA loop may be removed from polynucleotide 150, e.g., via chemical or enzymatic cleavage. Illustratively, the DNA loop may include a uracil which may be cleaved using USER enzyme.

FIG. 6 illustrates an alternative example in which a first portion 651 of polynucleotide 150 is coupled to a structure 650 that is at least partially located within the second fluidic compartment and retains the first portion of the polynucleotide in the second fluidic compartment. In operation (a) of FIG. 6, prior to the polynucleotide 150 being hybridized to the capture primer of particle 100, a first portion 651 of the polynucleotide is located in the second fluidic compartment 140 and a second portion 652 of the polynucleotide is located in the first fluidic compartment 130. Structure 650 may retain the first portion 651 of the polynucleotide 150 in the second fluidic compartment 140. For example, as illustrated in FIG. 6, the first portion 651 of the polynucleotide may be coupled (e.g., hybridized) to structure 650. In operation (b) of FIG. 6, when particle 100 is located at aperture 124 (e.g., by being carried past aperture 124 via flow of a fluid within first fluidic compartment 130), particle 100 captures polynucleotide 150 in a manner such as described with reference to FIGS. 1A-1H, e.g., via hybridization between polynucleotide 150 and a capture primer on particle 100. In operation (c) of FIG. 6, responsive to the polynucleotide 150 hybridizing to the capture primer, the first portion 651 of the polynucleotide is transported from the second fluidic 140 compartment into the first fluidic compartment 130 through first aperture 124. For example, as illustrated in operation (c) of FIG. 6, when polynucleotide 150 is hybridized to the capture primer of particle 100, force from the particle dissociates the first portion 651 of polynucleotide 150 from structure 650 and the structure remains within the second fluidic compartment 140. As illustrated in operation (d), aperture 124 is free to receive another polynucleotide 150 coupled to another structure 650 or the same structure 650.

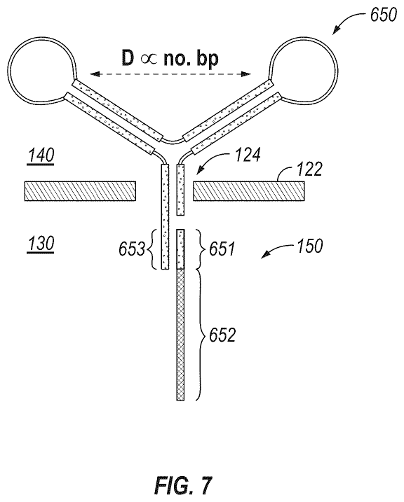

Any suitable structure 650 may be used to retain polynucleotide 150 at aperture 124, such as a DNA loop, a DNA hairpin, a cruciform folded double strand, or a dendrimer. In the nonlimiting example illustrated in FIG. 6, structure 650 includes a dendrimer including an oligonucleotide 653 having a sequence which is substantially complementary to, and which hybridizes to, first portion 651 of polynucleotide. In various examples, the dendrimer may include a polymeric (e.g., organic, inorganic, or hybrid organic-inorganic) backbone which is made via convergent or divergent routes, e.g., a backbone including a polypeptide, polyester, poly(amidoaminc), or poly(propyleneimine). FIG. 7 illustrates another nonlimiting example structure 650 that may be used in operations such as described with reference to FIG. 6, to retain polynucleotide 150 at aperture 124. In the example of FIG. 7, structure 650 includes a cruciform folded double stranded DNA similar to that described in the following references, the entire contents of each of which are incorporated by reference herein: Saccà et al., “DNA origami,” Angew. Chemic Int. Ed. 51:58-66 (2011); and Bikard et al., “Folded DNA in action: hairpin formation and biological functions in prokaryotes,” Microbiol. Mol. Biol. Rev. 74(4): 570-588 (2010). As illustrated in FIG. 7, the cruciform folded double stranded DNA includes oligonucleotide 653 having a sequence which is substantially complementary to, and which hybridizes to, first portion 651 of polynucleotide 150. Similarly as described with reference to operation (c) of FIG. 6, when polynucleotide 150 is hybridized to the capture primer of particle 100, force from the particle dissociates the first portion 651 of polynucleotide 150 from structure 650 and the structure remains within the second fluidic compartment 140. Similarly as described with reference to operation (d) of FIG. 6, aperture 124 then is free to receive another polynucleotide 150 coupled to another structure 650 or the same structure 650.

Note that oligonucleotide 653 and first portion 651 of polynucleotide may hybridize to one another in any suitable location, e.g., in the first fluidic compartment 130, the aperture 124, and/or in the second fluidic compartment 140. In the nonlimiting example shown in FIG. 7, oligonucleotide 653 and first portion 651 are located in the first fluidic compartment 130, such both the first portion 651 and second portion 652 of polynucleotide 150 are located in the first fluidic compartment. In comparison, the nonlimiting example shown in FIG. 6, oligonucleotide 653 and first portion 651 are located in the second fluidic compartment 140, such that the first portion 651 of polynucleotide 150 is located in the first fluidic compartment and the second portion 652 of polynucleotide 150 is located in the second fluidic compartment 140, such that polynucleotide 150 is partially translocated through aperture 124 while retained in the aperture by structure 650. In either example, structure 650 retains polynucleotide 150 at aperture 124 and inhibits another polynucleotide from entering the aperture until a particle captures the particular polynucleotide to which the structure 650 is coupled (e.g., through hybridization between oligonucleotide 653 and first portion 651). It will be appreciated that the number of complementary base pairs in oligonucleotide 653 and first portion 651 suitably may be selected so as to dissociate from one another under a force applied by the motion of particle 100. Optionally, structure 650 may remain within aperture 124 after being dissociated from polynucleotide 150. Optionally, such a structure may be removed from aperture 124 using a back-flushing operation, e.g., by flowing fluid at a suitable pressure through first fluidic compartment.

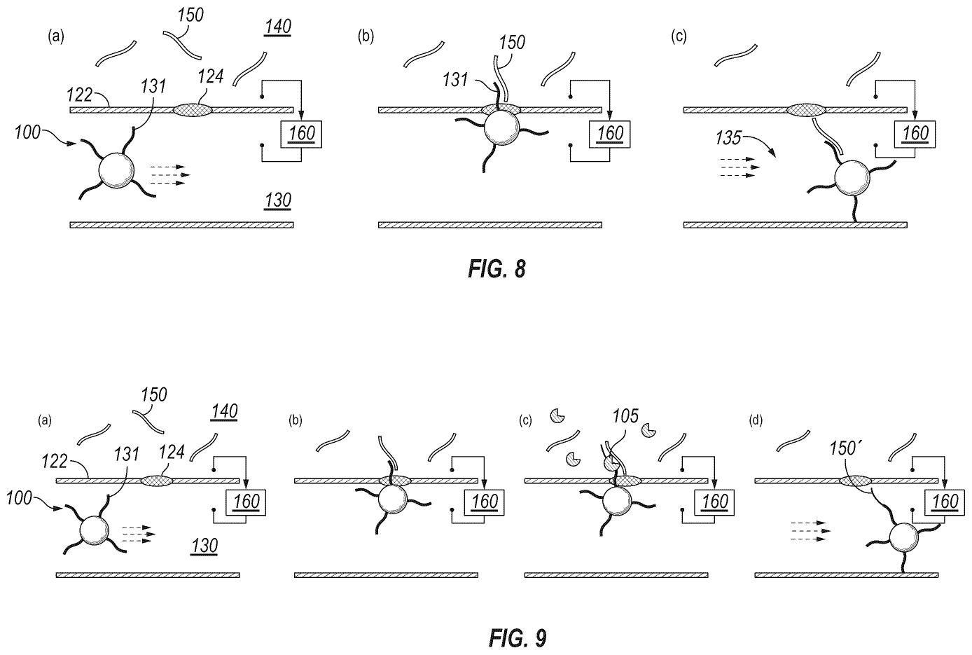

In yet other examples, controller 160 may transport the polynucleotide after the polynucleotide is hybridized to the capture primer. For example, FIG. 8 illustrates a configuration in which the capture primer of particle 100 extends through aperture 124 and hybridizes to the polynucleotide 150 in the second fluidic compartment 140. More specifically, at operation (a) of FIG. 8, particle 100 is located within first fluidic compartment 130 and transported to aperture 124 using controller 160 in a manner similar to that described with reference to FIGS. 1A-1H. Similarly as also described with reference to FIGS. 1A-1H, polynucleotides 150 are located within second fluidic compartment 140. As illustrated in operation (b) of FIG. 8, capture primer 131 (or equivalently capture primer 141) of particle 100 extends through aperture 124 and hybridizes to one of polynucleotides 150 at least partially within the second fluidic compartment 140. For example, adapter 154 of polynucleotide 150 may be complementary to, and may hybridize to, capture primer 131 similarly as described with reference to FIGS. 1A-1H, but in the second fluidic compartment rather than the first fluidic compartment. As illustrated in operation (c) of FIG. 8, when the polynucleotide 150 is hybridized to the capture primer 131, force from the particle 100 (e.g., exerted by the fluid 135 in which particle 100 is suspended) pulls the polynucleotide through the first aperture 124 and into the first fluidic compartment 130. The aperture 124 then may be reused to capture another polynucleotide 100 on another particle.

FIG. 9 illustrates an alternative configuration in which controller 160 may transport particle 100 within the first fluidic compartment 130 to the first aperture 124 so that the capture primer 131 (or 141) extends through the aperture to hybridize to the polynucleotide in the second fluidic compartment 140 such that an amplicon 151′ of the polynucleotide is generated in the second compartment and the amplicon is transported from the second fluidic compartment to the first fluidic compartment through the first aperture. More specifically, at operation (a) of FIG. 6, particle 100 is located within first fluidic compartment 130 and transported to aperture 124 using controller 160 in a manner similar to that described with reference to FIGS. 1A-1H. Similarly as also described with reference to FIGS. 1A-1H, polynucleotides 150 are located within second fluidic compartment 140. As illustrated in operation (b) of FIG. 9, capture primer 131 (or equivalently capture primer 141) of particle 100 extends through aperture 124 and hybridizes to one of polynucleotides 150 at least partially within the second fluidic compartment 140. For example, adapter 154 of polynucleotide 150 may be complementary to, and may hybridize to, capture primer 131 similarly as described with reference to FIGS. 1A-1H, but in the second fluidic compartment rather than the first fluidic compartment. As illustrated in operation (c) of FIG. 9, when the polynucleotide 150 is hybridized to the capture primer 131, a polymerase 105 in second fluidic compartment 140 extends that capture primer 131 based on the sequence of polynucleotide 150 so as to generate strand 150′ which is complementary to polynucleotide 150, and which is covalently coupled to particle 100. Polynucleotide 150 then optionally may be dehybridized from strand 150′. As illustrated in operation (d) of FIG. 9, force from the particle 100 (e.g., exerted by the fluid 135 in which particle 100 is suspended) pulls the polynucleotide through the first aperture 124 and into the first fluidic compartment 130. The aperture 124 then may be reused to capture another polynucleotide 100 on another particle.

In examples such as described with reference to FIGS. 8 and 9, controller 160 may use electrodes to apply potential differences between the first and second fluidic compartments 130, 140 so as to move the particle 100 to or from aperture 124, and/or so as to inhibit translocation of polynucleotides through aperture 124 that are not coupled to particle 100.

It will be appreciated that the present aperture-based devices and methods suitably may be adapted for use with a variety of fluidic architectures, including parallel channels, linear arrays, and/or planar arrays of apertures, such as apertures within nanopores. Illustratively, for high-throughput capture of polynucleotides on particles, an array of hundreds, thousands, or even millions of apertures (e.g., within corresponding nanopores) may be used, each such aperture being used capture a polynucleotide on a corresponding particle. In one nonlimiting example, the first fluidic compartment 130 may be loaded with first fluid 135 that includes polynucleotides, the second fluidic compartment 140 may be loaded with second fluid 145 that includes particles, and a first batch of capture events may be performed using these fluids and an array of apertures. The resulting seeded particles then may be removed, and the operations repeated to perform additional batches of capture events. Alternatively, the first and second fluids 135, 145 may be continuously flowed through the first and second fluidic compartments 130, 140 to sequentially perform capture events at each aperture of the array. In either example, any particles which are not seeded may be separated from particles which are seeded, and optionally may be recycled.

It will be understood that in nonlimiting examples such as described with reference to FIGS. 2-9, and in other examples that may be envisioned based on the present teachings, the polynucleotide which the particle captures subsequently may be amplified, e.g., using operations such as described with reference to FIGS. 1D-1G. Optionally, the resulting cluster of amplicons may be sequenced, for example by disposing the particle within a flowcell of a commercially available sequencing device, such as made by Illumina, Inc.

From the foregoing disclosure, it will be appreciated that primers and adapters having any suitable sequences may be used. In one nonlimiting example, amplification primers 131 are P5 amplification primers, and amplification primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters 155 may be full-length complementary P7 adapters (cP7) having the sequence 5′-TCGTATGCCGTCTTCTGCTTG-3′ (SEQ ID NO: 4), and are commercially available from Illumina, Inc.

It will be appreciated that various examples herein may be used with operations consistent with “bridge amplification” or “surface-bound polymerase chain reaction” and/or with other amplification modalities. One such amplification modality is “exclusion amplification,” or ExAmp. Exclusion amplification methods may further facilitate the amplification of a single target polynucleotide per particle and the production of a substantially monoclonal population of amplicons on a particle. For example, the rate of amplification of the first captured target polynucleotide using the present particles may be more rapid relative to much slower rates of transport and capture of target polynucleotides using the present particles. As such, the first target polynucleotide captured by the particle may be amplified rapidly and fill the entire particle, thus further inhibiting the seeding and/or amplification of additional target polynucleotide(s) by the particle. Even if a second target polynucleotide is captured at the particle after the first polynucleotide, the relatively rapid amplification of the first polynucleotide may fill enough of the particle to result in a signal that is sufficiently strong to perform sequencing by synthesis (e.g., the second region may be at least functionally monoclonal). The use of exclusion amplification may also result in super-Poisson distributions of particles including monoclonal clusters; that is, the fraction of particles in a collection of particles that are functionally monoclonal may exceed the fraction predicted by the Poisson distribution. Increasing super-Poisson distributions of useful clusters is useful because more functionally monoclonal particles may result in higher quality signal, and thus improved SBS.

Another method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present particles and methods may be adapted for use with recombinase to facilitate the invasion of the present amplification primers and orthogonal amplification primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp). For still further examples of amplification methods that are compatible with the present particles, see International Patent Application No. PCT/US2022/053005 to Ma et al., filed Dec. 15, 2022 and entitled “Hybrid Clustering,” and International Patent Application No. PCT/EP2023/058307 to Ma et al., filed Mar. 30, 2023 and entitled “Paired-End Resynthesis Using Blocked P5 Primers,” the entire contents of each of which are incorporated by reference herein.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of capturing a polynucleotide on a particle comprising a capture primer, the method comprising:
transporting the particle to a first aperture between a first fluidic compartment and a second fluidic compartment, wherein the particle is located in the first fluidic compartment, and wherein the polynucleotide is at least partially located in the second fluidic compartment;
transporting the polynucleotide from the second fluidic compartment to the first fluidic compartment through the first aperture; and
hybridizing the polynucleotide to the capture primer.

2. The method of claim 1, further comprising, after hybridizing the polynucleotide to the capture primer, transporting the particle away from the first aperture.

3. The method of claim 2, wherein transporting the particle to the first aperture and away from the aperture comprises flowing a fluid, in which the particle is suspended, through the first fluidic compartment and past the aperture.

4. The method of claim 1, further comprising synchronizing transport of the particle to the first aperture with transport of the polynucleotide through the first aperture.

5. The method of claim 4, wherein the synchronizing comprises:
detecting transport of the polynucleotide through the first aperture; and
controlling transport of the particle to the first aperture based on the detected transport of the polynucleotide through the first aperture.

6. The method of claim 4, wherein the synchronizing comprises:
transporting the particle through a second aperture and into the first fluidic compartment;
detecting transport of the particle through the second aperture; and
controlling transport of the polynucleotide through the first aperture based on the detected transport of the particle through the second aperture.

7. The method of claim 4, wherein the synchronizing comprises:
electronically controlling transport of the particle through a second aperture and into the first fluidic compartment; and
electronically controlling transport of the polynucleotide through the first aperture and into the first fluidic compartment.

8-12. (canceled)

13. The method of claim 1, wherein transporting the polynucleotide through the first aperture comprises flowing a fluid, in which the polynucleotide is suspended, through the aperture and into the first fluidic compartment.

14. (canceled)

15. The method of claim 1, wherein the polynucleotide is transported through the first aperture before hybridizing the polynucleotide to the capture primer.

16. The method of claim 15, wherein before being transported through the first aperture, the polynucleotide is located entirely in the second fluidic compartment.

17. The method of claim 1, wherein the polynucleotide is transported through the first aperture in response to hybridizing the polynucleotide to the capture primer.

18. The method of claim 17, wherein prior to hybridizing the polynucleotide to the capture primer, a first portion of the polynucleotide is located in the second fluidic compartment and a second portion of the polynucleotide is located in the first fluidic compartment.

19. The method of claim 18, wherein responsive to hybridizing the polynucleotide to the capture primer, the first portion of the polynucleotide is transported from the second fluidic compartment into the first fluidic compartment through the first aperture.

20. The method of claim 19, wherein the first portion of the polynucleotide retains the first portion of the polynucleotide in the second fluidic compartment.

21. The method of claim 20, wherein the first portion of the polynucleotide comprises a DNA loop, and wherein when the polynucleotide is hybridized to the capture primer, force from the particle dissociates the DNA loop.

22. (canceled)

23. The method of claim 20, wherein the first portion of the polynucleotide is coupled to a structure that is at least partially located within the second fluidic compartment and retains the first portion of the polynucleotide in the second fluidic compartment, and
wherein when the polynucleotide is hybridized to the capture primer, force from the particle dissociates the first portion of the polynucleotide from the structure, and the structure remains within the second fluidic compartment.

24. The method of claim 23, wherein the structure comprises a DNA loop, a DNA hairpin, a cruciform folded double strand, or a dendrimer.

25. (canceled)

26. The method of claim 1, wherein the polynucleotide is transported through the first aperture after hybridizing the polynucleotide to the capture primer.

27. The method of claim 26, wherein the capture primer extends through the aperture and hybridizes to the polynucleotide in the second fluidic compartment.

28. The method of claim 27, wherein when the polynucleotide is hybridized to the capture primer, force from the particle pulls the polynucleotide through the first aperture and into the first fluidic compartment.

29. The method of claim 1, wherein the polynucleotide comprises an adapter that is complementary to the capture primer.

30-31. (canceled)

32. The method of claim 1, wherein the polynucleotide is single-stranded.

33. The method of claim 1, wherein the polynucleotide is double-stranded.

34-35. (canceled)

36. A method of generating a clonal cluster of a polynucleotide on a particle, the method comprising:
capturing the polynucleotide on the particle using the method of claim 1; and
using a plurality of amplification primers on the particle to amplify the polynucleotide.

37-72. (canceled)


SEQUENCE LISTING (4 sequences)

SEQ ID NO: 1 - DNA, 20 bp, synthetic construct
  source 1..20: mol_type = other DNA; note = P5 amplification primer
aatgatacggcgaccaccga

SEQ ID NO: 2 - DNA, 21 bp, synthetic construct
  source 1..21: mol_type = other DNA; note = P7 amplification primer
caagcagaagacggcatacga

SEQ ID NO: 3 - DNA, 20 bp, synthetic construct
  source 1..20: mol_type = other DNA; note = complementary P5 adapter
tcggtggtcgccgtatcatt

SEQ ID NO: 4 - DNA, 21 bp, synthetic construct
  source 1..21: mol_type = other DNA; note = complementary P7 adapter
tcgtatgccgtcttctgcttg